(19) United States
(12) Patent Application Publication
(10) Pub. No.: US 2021/0223224 A1
(43) Pub. Date: Jul. 22, 2021

(54) MEAT QUALITY ANALYZER

(71) Applicant: Texas State University, San Marcos, TX (US)

(72) Inventor: Carl R. Richardson, Kyle, TX (US)

(73) Assignee: Texas State University, San Marcos, TX (US)

(21) Appl. No.: 17/121,520

(22) Filed: Dec. 14, 2020

Related U.S. Application Data
(60) Provisional application No. 62/964,429, filed on Jan. 22, 2020.

(51) Int. Cl. G01N 33/12; G01N 27/12

(52) U.S. Cl. CPC G01N 33/12; G01N 27/121

(57) ABSTRACT

Embodiments of the present disclosure pertain to methods of evaluating a meat sample by measuring electricity conducted from water in the meat sample and correlating the evaluation to a property of the meat sample. The water forms (e.g., free water, immobilized water, and bound water) can be evaluated by measuring electricity conducted from water in the meat sample. The property of the meat sample can include, without limitation, a color of the meat sample, or a tenderness of the meat sample.

### CROSS-REFERENCE TO RELATED APPLICATIONS

This application claims priority to U.S. Provisional Patent Application No. 62/964,429, filed on Jan. 22, 2020. The entirety of the aforementioned application is incorporated herein by reference.

### BACKGROUND

Current methods and systems for evaluating the quality of meat have numerous limitations. Various embodiments of the present disclosure aim to address such limitations.

### SUMMARY

In some embodiments, the present disclosure pertains to methods of evaluating a meat sample by measuring electricity conducted from water in the meat sample and correlating the measurement to a property of the meat sample. In some embodiments, the water is derived from at least one water form. In some embodiments, the at least one water form includes, without limitation free water, immobilized water, bound water, or combinations thereof.

In some embodiments, measuring electricity conducted from water in a meat sample occurs by measuring electron flow from resulting water in the meat sample. In some embodiments, the measuring occurs by measuring an electrical conductivity of the meat sample.

In some embodiments, the meat sample includes, without limitation, beef, poultry, fish, pork, and combinations thereof. In some embodiments, the meat sample is beef.

In some embodiments, the evaluated property of the meat sample is a color of the meat sample. In some embodiments, the evaluated property of the meat sample is tenderness of the meat sample.

### FIGURES

FIGS. 1A and 1B illustrate methods of evaluating properties of a meat sample in accordance with various embodiments of the present disclosure.

FIG. 2 provides an illustration of a multimeter for evaluating meat quality in accordance with various embodiments of the present disclosure.

FIGS. 3A and 3B provide images of an R-EC meat analyzer box (R-EC Meat Box) for evaluating meat quality in accordance with various embodiments of the present disclosure.

FIGS. 4A, 4B, and 4C illustrate the concept of water forms in muscle proteins, and the effects of pH on meat related to excess, neutral, or negative charges. The description of water forms shown has been generally understood. However, understanding how to measure and apply information on water activity in meat for the purpose of quality assessment of tenderness and color has not been demonstrated. From: The Science of Meat and Meat Products, 2nd ed. J. F. Price and B. S. Schweigert, eds. W. H. Freeman and Co., San Francisco. 1971.

FIG. 5 illustrates a color comparison across fresh strip loins.

FIG. 6 illustrates a tenderness comparison across cooked strip loins.

FIG. 7 illustrates a relationship of electrical conductivity to color in beef loins.

FIG. 8 illustrates a relationship of electrical conductivity to tenderness in beef loins.

FIG. 9 illustrates a relationship of fresh electrical conductivity to tenderness in beef loins.

FIG. 10 illustrates a relationship of cooked electrical conductivity to color in beef loins.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The meat industry, particularly the beef industry, faces major challenges due to the broad-ranging variability of raw material, which ultimately translates into high variability in product quality and low process control over the commercialized end product. Meat quality and consistency are important traits that are mostly governed by combination of animal genetics, rearing and nutritional status. These factors affect the fat, lean and connective tissue components of meat and therefore influence meat quality (i.e., “functional” characteristics that range from sensory-domain taste and appearance properties and eventual customer satisfaction).

The overall consumer experience for the quality of meat is determined by a combination of tenderness, flavor and juiciness with individual contribution for 43.4%, 49.4%, and 7.4%, respectively (P<0.05; R₂>0.99). The palatability and social factors, which include tenderness and color, are important selection criteria used by consumers for beef. However, existing analytical methods to determine nutritional value of meat samples (e.g., beef steaks) are unable to distinguish between tenderness values (e.g., tough or tender) or color scores (e.g., dark, bright, or light). For instance, beef industry and animal and meat scientists over the past century (i.e., since 1926) have relied upon USDA grading systems and color scores as primary factors in evaluation of beef and consumer acceptance.

In addition, the description of water forms in meat (i.e., free, immobilized, and bound) as related to their orientation toward charged properties and pH was published in 1971 (Wisner-Pedersen, J., “Chemistry of Animal Tissues-Water,” in The Science of Meat Products, 2^{nd} ed. J. F. Price and B. S. Schweigert, eds. W.H. Freeman and Co. San Francisco. Copyright 1971). However, no research data have been reported on the effects of water form on tenderness and color of beef or other meats.

As such, a need exists for more effective systems and methods for evaluating the quality of meat. Various embodiments of the present disclosure address the aforementioned needs.

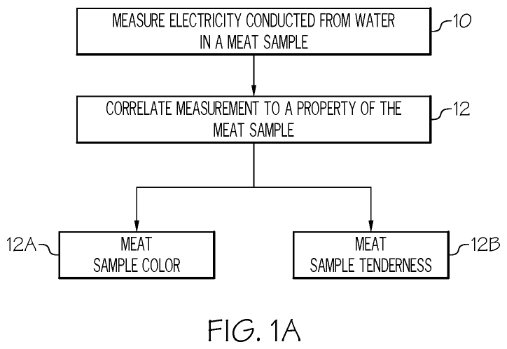

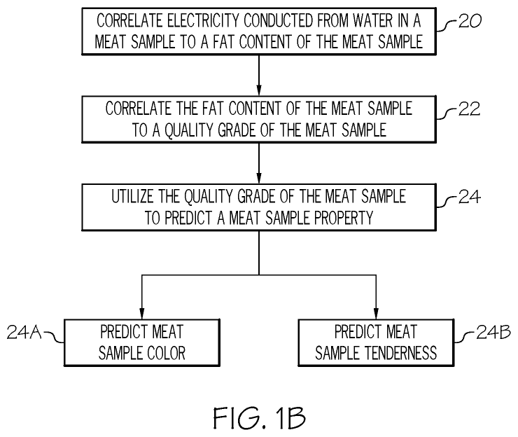

In some embodiments, the present disclosure pertains to methods of evaluating a meat sample. In some embodiments illustrated in FIG. 1A, the methods of the present disclosure include steps of: measuring electricity conducted from water in the meat sample (step 10); and correlating the measurement to a property of the meat sample (step 12), such as meat sample color (step 12A) or meat sample tenderness (step 12B). In some embodiments, the water is derived from at least one water form.

As set forth in more detail herein, the present disclosure may utilize various methods to measure electricity conducted from water in various meat samples. The present disclosure may also utilize various methods to correlate the measurements to various meat properties.

Water Forms

Various water forms may be associated with a meat sample. For instance, in some embodiments, the water forms include free water, immobilized water, bound water, or combinations thereof.

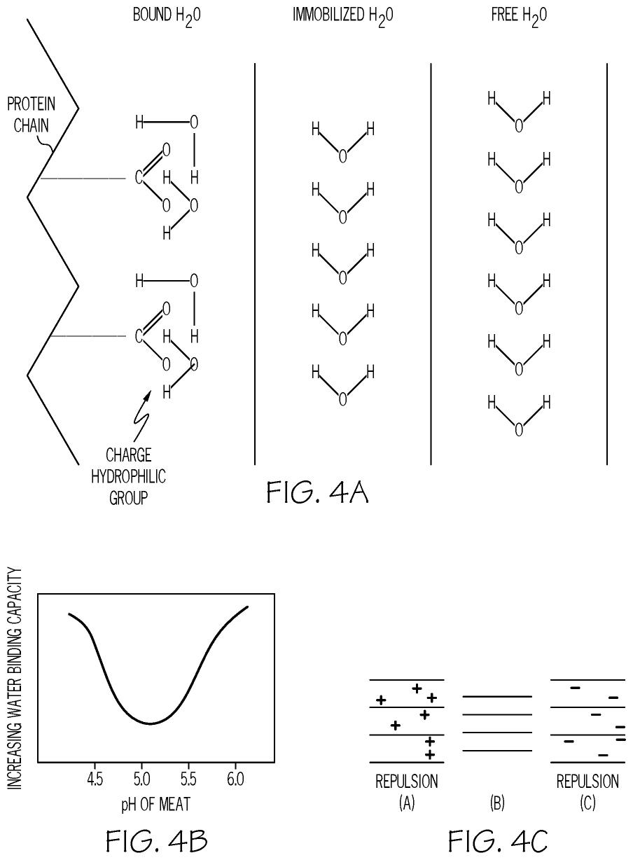

As used herein, free water generally refers to water that is not associated with any other elements or compounds. In some embodiments, free water refers to water that is not bound or attracted to the meat sample (see, e.g., right panel of FIG. 4A). In some embodiments, the free water may be in the form of H₂O that is in equilibrium with OH⁻ and H₃O⁺.

Free water consists of two elements: hydrogen and oxygen. In some embodiments, free water forms the chemical compound hydrogen oxide. In some embodiments, the chemical formula of free water is H₂O to form a liquid. In some embodiments, free water is shaped with oxygen in the center and hydrogen on either side at an angle of 104.5°.

In some embodiments, free water reacts only within its chemical moiety. In some embodiments, a portion of free water molecules splits into the chemically active compounds of OH⁻ and H⁺ while the rest is in the H₂O chemical structure.

As used herein, immobilized water generally refers to water that is entrapped when the charged components of free water (OH⁻ and H⁺) become chemically attracted to other elements or compounds. In some embodiments, immobilized water refers to water that is not bound to the meat sample but attracted to the meat sample (see, e.g., middle panel of FIG. 4A). In some embodiments, the immobilized water may also be in the form of H₂O that is in equilibrium with OH⁻ and H₃O⁺. In some embodiments, the immobilized water may be attracted to the meat sample through hydrogen bonds between H₂O molecules and moieties on the meat sample. In some embodiments, the immobilized water may be attracted to the meat sample through ionic bonds between OH⁻ molecules and cationic moieties on the meat sample. In some embodiments, the immobilized water may be attracted to the meat sample through ionic bonds between H₃O⁺ molecules and anionic moieties on the meat sample.

As used herein, bound water refers to a portion of free water that has been chemically dissociated to form the ionic parts OH⁻ and H⁺, which attach to and become part of the chemical structure of a muscle. In some embodiments, bound water refers to water that is bound to the meat sample (see, e.g., left panel of FIG. 4A). In some embodiments, the bound water may also be in the form of H₂O that is in equilibrium with OH⁻ and H₃O⁺. In some embodiments, the bound water may be bound to the meat sample through hydrogen bonds between H₂O molecules and moieties on the meat sample. In some embodiments, the bound water may be bound to the meat sample through ionic bonds between OH⁻ molecules and cationic moieties on the meat sample. In some embodiments, the bound water may be bound to the meat sample through ionic bonds between H₃O⁺ molecules and anionic moieties on the meat sample.

In some embodiments, bound water may no longer be liquid. In some embodiments, bound water may be referred to as hydrated water or non-reactive water.

Measuring Electricity Conducted from Water

Various methods may be utilized to measure electricity conducted from water in the meat sample. For instance, in some embodiments, the measurement occurs by measuring micro Siemens of electricity conducted from water in the meat sample. In some embodiments, the measurement occurs by measuring electron flow from the water in the meat sample.

In some embodiments, the measurement occurs by measuring an electrical conductivity of the meat sample. Various methods may be utilized to measure an electrical conductivity of a meat sample. For instance, in some embodiments, the electrical conductivity of a meat sample is measured by measuring electrical current in a meat sample. In some embodiments, the electrical current in meat sample is measured by placing the meat sample in a container with water (e.g., deionized water) prior to the measurement of electrical current. Thereafter, the container is connected to one or more electrodes for measuring electrical conductivity (i.e., the electrical current in the meat sample).

In some embodiments, the meat sample is emulsified in a blender prior to the measurement of electrical current. In some embodiments, the meat sample is cooked prior to the measurement of electrical current. In some embodiments, the meat sample remains raw during the measurement of electrical current.

In some embodiments, the overall electrical conductivity produced and measured from meat samples is derived from water activity in the meat sample. In some embodiments, the water activity in the meat sample is derived from free water, immobilized water, bound water, or combinations thereof.

Meat Samples

The methods of the present disclosure can be utilized to evaluate various meat samples. For instance, in some embodiments, the meat sample includes, without limitation, beef, poultry, fish, pork, and combinations thereof.

In some embodiments, the meat sample is beef. In some embodiments, the beef includes, without limitation, beef loins, beef jerky, cooked beef, uncooked beef, fresh strip loins, or combinations thereof.

Correlation of Measurements to a Property of a Meat Sample

Various methods may also be utilized to correlate measured electricity conducted from water in a meat sample to meat sample properties. For instance, in some embodiments, the correlating includes comparing the measured electricity conducted from water in the meat sample to known properties associated with the measured electricity conducted from water in similar meat samples. In some embodiments, the comparing occurs by comparing the measured electricity conducted from water in the meat sample to a database that includes the known properties.

In some embodiments illustrated in FIG. 1B, the measured electricity conducted from water in a meat sample is first correlated to a fat content of a meat sample (step 20). Thereafter, the fat content of the meat sample is correlated to a quality grade of the meat sample (step 22). For instance, in some embodiments, a lower fat content represents a lower quality grade of a meat sample and a higher fat content represents a higher quality grade of a meat sample.

Next, the quality grade of the meat sample is utilized to predict a meat sample property (step 24). For instance, in some embodiments, the quality grade of the meat sample is utilized to predict the color of the meat sample (step 24A). In some embodiments, the quality grade of the meat sample is utilized to predict the tenderness of the meat sample (step 24B).

Various methods may also be utilized to correlate measured electricity conducted from water in a meat sample to fat content, quality grade, and meat sample properties. For instance, in some embodiments, the correlating includes comparing the measured electricity conducted from water in the meat sample to known fat contents, quality grades, and meat sample properties associated with the measured electricity conducted from water in similar meat samples. In some embodiments, the comparing occurs by comparing the measured electricity conducted from water in the meat sample to a database that includes the known fat contents, quality grades, and meat sample properties.

In some embodiments, the correlating occurs by utilizing an algorithm to correlate the measured electricity conducted from water in the meat sample to one or more properties of the meat sample. In some embodiments, the algorithm includes a machine learning algorithm.

The measured electricity conducted from water in meat samples can be correlated to numerous meat sample properties. For instance, in some embodiments, the correlated property of the meat sample is a color of the meat sample. In some embodiments, the color of the meat sample represents a color score of a cooked or an uncooked meat sample. In some embodiments, the color score includes dark, bright, or light.

In some embodiments, the color can be characterized by a color variable. In some embodiments, the color variable includes, without limitation, variables of chroma, hue, L*, a*, b*, or combinations thereof.

In some embodiments, the correlated property of the meat sample is tenderness of the meat sample. In some embodiments, the tenderness of the meat sample represents the tenderness of the meat sample in cooked or uncooked form. In some embodiments, the tenderness of the meat sample can be characterized by a tender value in kg/f.

Without being bound by theory, water activity in meat samples (e.g., from free water and immobilized water) corresponds to tenderness and meat color in two separate physio-chemical ways. One, water activity is inversely correlated to tenderness because water activity decreases as quality grade (e.g., fat content) increases and flavor and juiciness increases. Two, water activity is correlated to color because water activity changes the reflectance of colors in meat samples. In some embodiments, as the fat content of a meat sample changes, electrical conductivity changes due to differences in water activity.

Applications and Advantages

The methods of the present disclosure can have numerous advantages and applications. For instance, in some embodiments, the methods of the present disclosure can provide value to the food industry and to meat consumers through improved quality assessment, which is greatly needed in order to more accurately predict tenderness, and to evaluate color attributes.

In some embodiments, the methods of the present disclosure can be utilized to help improve quality appraisal of meat samples, such as beef steaks and cuts. In some embodiments, the methods of the present disclosure provide a more accurate and reliable prediction of meat tenderness and a new procedure to appraise meat color.

The methods of the present disclosure also provide numerous advantages over the primary worldwide methods in use for meat quality evaluation. In particular, prior meat evaluation methods have been based on animal maturity and marbling (i.e., intramuscular fat). However, unlike the methods of the present disclosure, the aforementioned methods do not take into consideration the configuration (i.e., form) of water in the muscle tissue and its relationship to tenderness, color and overall consumer acceptance.

Moreover, because of the complex chemical structure of meat (i.e., muscle, fat, and water), which is the result of animal genetics, animal age, and nutritional feeding management, the methods of the present disclosure will help improve overall quality appraisal of meat (e.g., beef steaks and other retail cuts of beef).

Published research by many scientists over several decades have revealed noteworthy information on “dark cutters” (dark cutting beef) and “PSE pork” (pale, soft, and exudative). These negative physiological changes in muscle of these two food species result from abnormal metabolism of water, which is tied to genetic origin and stress prior to harvesting. As such, in some embodiments, the systems and methods of the present disclosure could also be of value in focusing more attention on water metabolism tied to these physiological disorders and in finding possible ways to prevent them. In particular, the systems and methods of the present disclosure can solve two-fold problems: (1) more accurate and reliable prediction of beef tenderness; and (2) a new procedure to appraise meat color.

### Additional Embodiments

Reference will now be made to more specific embodiments of the present disclosure and experimental results that provide support for such embodiments. However, Applicants note that the disclosure herein is for illustrative purposes only and is not intended to limit the scope of the claimed subject matter in any way.

### Example 1. Utilization of the R-EC Meat Analyzer Box (R-EC Meat Box) to Characterize Beef

This Example illustrates the characterization and analysis of water forms (free, immobilized, and bound) in beef by measuring the micro Siemens of electricity conducted from water activity in beef samples. The evaluation was performed by measuring electron flow from resulting water chemical activity in beef samples. This Example provides a way to use electrical conductivity (EC) determinations to estimate or predict tenderness of cooked beef, and to determine color scores of fresh beef.

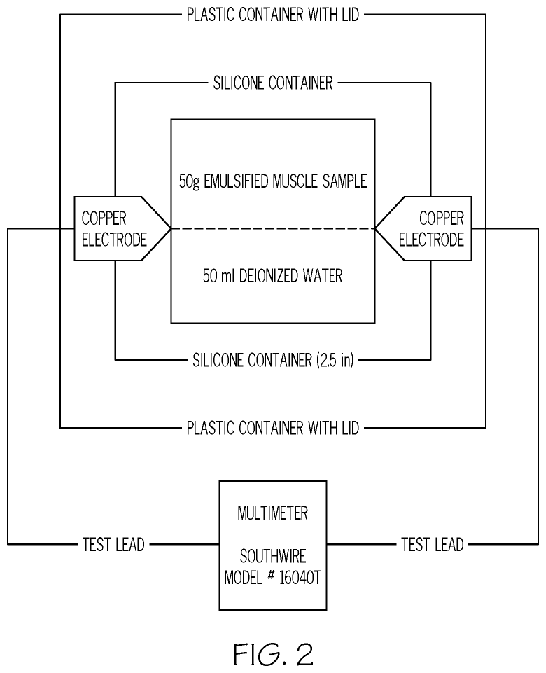

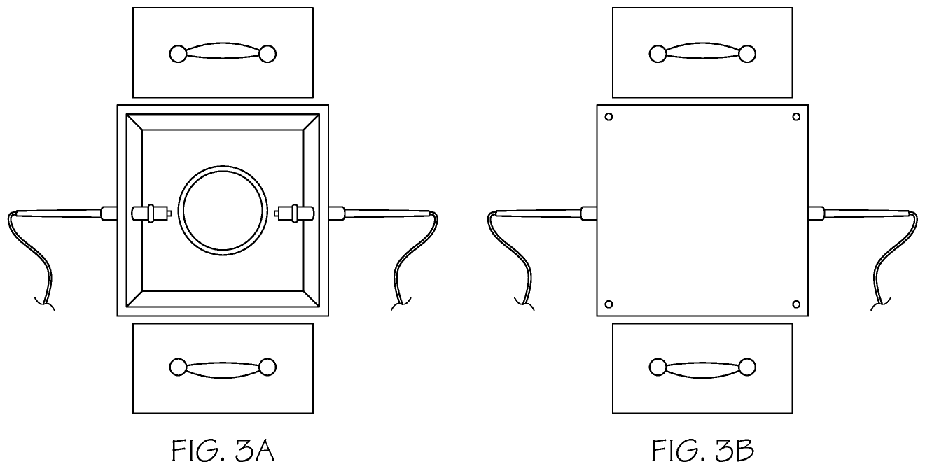

The R-EC Meat Analyzer “Box” (“R-EC Meat Box”, illustrated in FIGS. 2, 3A and 3B) was utilized for the measurements in this Example. The R-EC Meat Box is a device and a procedural process involving the measurement of electrical conductivity (EC) of meat samples.

The R-EC Meat Box characterized and analyzed water forms in meat by measuring the micro Siemens of electricity conducted by water activity in meat samples. The invention was developed to use EC to describe and statistically define the configuration (forms) of water in fresh and cooked beef loin samples, and ground beef, in order to improve methods used for quality assessment of beef.

The R-EC Meat Box measures EC arising from “free water” and “immobilized water” in meat samples. The third form of water in meat is “bound water” and it can be calculated by difference from determination of the other two forms.

Only beef was studied in this Example, but application to other types of meat is feasible. Table 1 provides a hypothetical example of how beef or beef steaks could be characterized using current industry criteria in conjunction with EC values.

TABLE 1
Hypothetical example of how beef or beef steaks could be characterized
using current industry criteria in conjunction with EC values.

| | | | Fresh EC | Cooked EC |
|---|---|---|---|---|
| Steak type | Grade | Color | value | value |
| Strip loin | Select | Bright cherry red | 6.00 | 5.50 |
| Strip loin | Choice | Bright cherry red | 4.00 | 5.00 |
| Strip loin | Prime | Bright cherry red | 2.00 | 4.50 |

### Example 1.1. Electrical Conductivity and Cooking Loss of Beef Loins and Ground Beef

Appraisal of beef tenderness and overall quality assessment by consumers may be variable. In this Example, two sources of beef loins and ground beef were used to determine if electrical conductivity measurements (ECM) are correlated with tenderness and cooking loss. Source 1 (S-1) was Choice grade loins and 80:20 ground beef, and Source 2 (S-2) was Prime grade loins and 80:20 ground beef. Steaks were cooked at 93° C. in a smoker without humidity or smoke to an internal temperature of 71° C. Warner-Bratzler shear force values (WBSF) were determined on steaks. Samples of both raw and cooked loins (n=48), and ground beef (n=80) were emulsified for ECM.

Procedures used for ECM consisted of using emulsified samples enclosed in a silicon vessel and concealed in a plastic bowl with a lid and a hole in two sides, for connecting embedded copper electrodes to a digital multimeter. See FIGS. 3A-3B. ECM was measured in micro Siemens (pS) over 60 second periods with a sampling rate of two per seconds.

Cooking loss was measured after cooking to 71° C. in George Forman grills. The surface area of ground beef patties was determined using the equation SA_{cylinder}=2πr²+2πr²h. Data analyses used were Pearson correlations, regression, and paired t-tests. Results show raw loin steaks from S-2 had higher ECM than S-1, 8.45 and 3.12 pS, respectively (P<0.01, r=0.74).

ECM values of cooked loin steaks were numerically higher for S-2 but was not different from S-1 (P>0.05). In addition, cooking loss was not different between sources (P>0.05). Raw and cooked ECM values were significantly correlated (P<0.01). S-2 burger patties had lower cooking loss and muscle shrinkage than S-1 (P<0.05). These results indicate that ECM can be used as a quality measurement of beef loins and ground beef, and ECM is correlated with WBSF tenderness values.

### Example 1.2. Quality Grading Using Color and Electrical Conductivity of Beef

Four sources of beef were used to determine if the degree of variance in color (Hunter LH) and electrical conductivity (EC) measures correlate to quality grade. It also tested the reliability of the Varian spectrophotometer machine for color scores in beef. Source 1 (S1) samples varied in cuts that were frozen and then thawed. S2-S4 were delivered whole and cut into 16 individual, 1-inch thick samples. Source 2 (S2) was whole boneless ribeye and choice grade. Source 3 (S3) was a whole strip loin, Wagyu bred and prime grade. Source 4 (S4) was a whole strip loin, Akaushi breed and prime grade.

All color measurements (n=48) were taken on raw samples. EC (n=48) was measured in micro Siemens (pS) for 120 s with sampling rate two measures per s. An emulsified meat slurry solution was placed into a 3 inch wide silicon vessel with copper electrodes attached to both sides, and placed into plastic container connected to a digital multimeter. See FIGS. 3A-3B. Analysis of variance (ANOVA) was used for data analysis on color & EC replications and total color &EC means and performed across 51 and then S2-S4.

Color results across color mean replications for individual samples were non-significant (P>0.05, 95% confidence level). Source color means for S1=33.6873, S2=55.4319, S3=47.8122, and S4=53.5633. SI had the lowest LH mean compared to S2-S4 and presumed to be a result of myoglobin state. These data indicate reliability of the Varian color machine as a method for scoring steaks. EC results showed prime grades S3 and S4 had lower scores compared to 51 and S2 with EC means of S1=24.8043, S2=63.5294, S3=17.0451, and S4=18.9656. S3 and S4 both had significant correlations (P<0.05) between EC and color scores indicating EC and color correlations can be useful in quality grading of beef.

### Example 1.3. Discussion

Appraisal of beef tenderness and overall quality assessment by consumers may be variable. Different sources of beef loin steaks and ground beef were studied in this Example to determine if EC measurements are correlated with tenderness and cooking loss. Results from three different studies in Applicants' lab show that EC measurements are different (P<0.05) between choice and prime quality grades of commercial beef. One study in which beef loin color was evaluated, it was found that EC measurements are correlated (P<0.05) with color of beef. Overall results of these studies indicate that EC measurements can be used in quality assessment of beef loins and ground beef, and EC is correlated with shear force tenderness values of cooked steaks.

### Example 1.4. Steps Involved in Electrical Conductivity Measurements

The steps involved in the electrical conductivity measurements illustrated in this Example 1 can have numerous embodiments. The following are exemplary steps:

1. Obtain beef strip loins of known USDA Quality Grades for water activity testing.
2. Obtain a commercially available digital multimeter that measures electrical conductivity (EC) in micro Siemens.
3. Select 50-gram samples of fresh beef strip loin steaks, or other beef cuts for testing.
4. Emulsify each 50-gram sample of fresh beef with 50 milliliters of deionized water in a blender to obtain a uniform slurry.
5. Cook strip loin steaks for a set time and temperature to use in determining tenderness by shear force measurements.
6. Determine tenderness of core samples taken from cooked steaks.
7. Composite 50-gram samples from beef cores for testing.
8. Emulsify each 50-gram sample of cooked beef with 50 milliliters of deionized water in a blender to obtain a uniform slurry.
9. Place fresh beef slurry or cooked beef slurry in the electrical conductivity (EC) sample container.
10. Secure lid on the EC sample container.
11. Connect the positive and negative test leads from the multimeter to the copper electrodes on opposite sides of the EC sample container.
12. Connect the multimeter via an app to an iPad or computer.
13. Leave the digital multimeter test leads connected to the EC sample container electrodes for a continuous 60 second period (one minute). Electrical conductivity sampling rate of the digital multimeter is two per second for a total of 120 per minute.
14. Between sample EC determinations, empty the sample container and rinse it with deionized water.
15. Keep laboratory EC testing area free from air and equipment vibrations, such as, from exhaust fans, leaning on counter or table where a meat analyzer is placed.

### Example 1.5. References

M. S. Thesis by S. P. Martinez titled PREDICTING WATER HOLDING CAPACITY AND FORMS OF WATER LOSS IN BEEF SOURCES, Texas State University. May 2017.
M. S. Thesis by M. S. Ramkumar titled THE CORRELATION BETWEEN COLOR AND ELECTRICAL CONDUCTIVITY IN BEEF, Texas State University. December 2017
Thesis by C. S. Mesquita titled EFFECTS OF WATER CONFIGURATION (FORM) IN BEEF LOINS ON COLOR, TENDERNESS, AND ELECTRICIAL CONDUCTIVITY, Texas State University. May 2020.

### Example 2. Effect of Water Configuration (Form) in Beef Loins on Color, Tenderness and Electrical Conductivity

In this Example, beef strip loins were used to study about the effects of apparent water configurations (i.e., free, immobilized, and bound) on color, electrical conductivity, and tenderness. These variables contribute to consumer sensory attributes that effect quality factors in beef consumption. Color variables (n=36) tested involved chroma, hue, L*, a*, and b* to determine correlation across fresh strip loins. The Pearson correlation data demonstrated a correlation at (P<0.0001) between chroma, hue, L*, a*, and b* across all loins.

Further analysis displayed similar conclusions to contain a significant difference. Beef jerky (n=72), cooked, and fresh strip loins (n=36) were evaluated for data analysis using electrical conductivity measurements (ECM). Beef jerky determinations displayed high electrical conductivity (EC) with (SEM=5.05), and ECM was inconclusive based on ingredients listed (food additives). Cooked strip loins, across strip loins, resulted in low ECM (P=0.37) indicated no significant difference. Fresh strip loins were compared among strip loins and indicated a trend (P<0.078). Both fresh and cooked strip loins were compared, and ECM showed a three-fold increase across loin types (P<0.0001). Tenderness (n=216) was different (P<0.0001) across strip loins. All data were collected on one source of beef strip loins that came from different animals.

Data analyses were conducted through SAS software using Pearson correlations and ANOVA to determine correlation and comparison across strip loins. Results of these experiments show that there are correlations between EC and color of fresh beef loins, and between EC and tenderness of cooked beef loins.

### Example 2.1. Experimental Design for Electrical Conductivity of Beef Jerky

An EC experiment was completed on four water sources, and deionized water was chosen for further analysis based on its low reactivity. The beef jerky was acquired from a well-known commercial source, representing a specific form of water configuration. Based on the ingredients list on the jerky, food additives or other considerations were added to the beef products.

This design began with the removal of beef jerky from 11 bags, each weighing 16 ounces. The electrical conductivity of beef jerky compromised of 50 g samples of beef jerky and was evaluated for EC measurement (n=72 beef jerky samples×1 replication=72 total observations). Each beef jerky strip was cut into square pieces approximately 1 centimeter in diameter. Upon completion, a total of 72 medium-sized square pieces of brown butcher paper was cut and labeled with a permanent marker from 1-72.

Each square piece of butcher paper started at number 1. 50 g of beef jerky was weighed and put onto the paper and set aside. This procedure was repeated 72 times to provide uniformity. After each sample was weighed, it was placed in 50 ml of deionized water and put into a Ninja BL456 blender and emulsified. Once emulsified, the meat slurry was transferred into a silicon vessel.

Two copper electrodes were on the opposite ends of the silicon container to ensure accurate EC measurements. An EC measurement continued for 60 seconds with the applied rate of 2 readings per second. Voltage and current flow were monitored through a multimeter (16040T True RMS Multimeter, Southwire Tools & Equipment). Between the sample readings, all materials and equipment exposed were rinsed with deionized water after every trial run. The trial runs consisted of 72 replications of EC measurements of commercial beef jerky to test ionic strength. The beef jerky analysis was necessary for testing ionic strength in beef jerky to evaluate the ionic strength of bound water.

### Example 2.2. Experimental Design for Color in Fresh Steaks

Beef loin samples developed for color measurements were procured from one source of six full, fresh strip loins of the same USDA grade. To ensure uniformity on color evaluations, the American Meat Science Association (AMSA) Color Measurement Guidelines provided a standard on each parameter for color values. The source used was of prime quality grade brand named HeartBrand Beef known for the Akaushi breed of cattle procured from a local retail rancher. The source of beef loins was prime grade from a local, commercial, retail rancher.

The beef loins arrived at Texas State University-San Marcos's meat laboratory as fresh whole loins. Each loin was cut into six steaks per loin, one-inch thick. All steaks were prepared with the same procedures for color evaluation. Before color measurements were taken, each steak was placed on brown butcher paper and a plastic wrap over each steak. The plastic wrap ensured a reduced oxygen exposure on the surface and provided a standardized procedure for calibration. During this time, each of the steaks remained on a sterilized meat processing table for 30 minutes to adjust to room temperature.

Thereafter, the color collection of data commenced, and continued until all measurements were concluded. Steaks were measured using the Varian Cary 50 Series Spectrophotometer following several parameters per AMSA to ensure standardization. All steaks were set to Illuminant A (this illuminant detects red wavelengths efficiently), observer angle of 10° (capturing a substantial sample portion), a wavelength of 830 to 360 nm with a scan range of 1 nm (that reflects a definite myoglobin percentage present on the meat surface) and an aperture size of 1.5 mm in diameter (adjusted per sample size). In conjunction, the spectrophotometer has an extended device known as the Harrington Barrelino device that is connected to the spectrophotometer to assist light scattering projection essential for data collection.

Reflectant score parameters were measured to 0%-100% established by the reflectance standard. Specific locations on the steak were tested and measured with a one inch diameter cookie cutter that reflected the accuracy for the measurement of values for electrical conductivity and tenderness. The reported color scores in this study used Commission Internationale de l'Eclairage (CIE) L*a*b* that determined the values for lightness, redness, and yellowness color of the steak. Calibration for each group reflected on a 0%-100% reflectance score and was completed through a white tile calibration piece originated with a spectrophotometer. Calibration standard for each steak was set with the white tile placed on a flat surface with a clear plastic wrap. Next, the Barrelino device was placed on top to demonstrate a baseline value for the scanned steaks. Prior to the collection of data, calibration was initially performed. This technique ensured proper adjustments to verify steaks were appropriately measured in the time frame allotted.

Several other parameters were needed to standardize the score, such as “Y Mode” equivalent to the % R (reflectance), “Av” Time (s) of 0.0125 (time used to calculate a data value) conjoined to a dual-beam mode and Delta LAB tolerance of 5.00 “E” (related to a change in time). The spectrophotometer was selected for Delta LAB tolerance E, which referred to a change in time, not indicative of this study.

Several corrections were chosen based on thickness and refractive index in the settings section. This study consisted of Thickness [Known % T] and Thickness [unknown % T] set at 1.00. Color reports were analyzed to specifically focus on Autoconvert ASCII (csv) with log a conversion tool for data importation. Each steak was scanned at 2 locations with 3 scans per location. Each of the values for the area was averaged to attain a mean color value for each location and a final average per loin.

All values were statistically analyzed (n=6 loins×6 steaks×2 locations/steak=72 total observations). Furthermore, steak color values were captured, beef loins were weighed individually using a food-grade scale, and the room temperature was determined through a mercury glass thermometer encased in wood to provide uniformity. All steaks were placed into gallon-sized Ziploc bags labeled corresponding to the loin and steak number using a permanent marker, placed into a white plastic bin, and stored into the walk-in cooler at 34° F. (1° C.) overnight. Color measurements were taken to provide an improved appraisal of overall true color value in beef loins in addition to visual appraisal.

### Example 2.3. Experimental Design for Electrical Conductivity of Fresh Steaks

After being stored overnight, all steaks were removed from the walk-in cooler and placed onto butcher paper labeled with the corresponding loin and steak number. All steaks were allowed to rest for one hour before EC experimentation. After rest, a 50 g sample of each raw beef steak was extracted using a fillet knife and weighed using a food-grade scale. The raw beef loin 50 g samples were placed in pint-size Ziploc bags labeled with a permanent marker according to the loin and sample number.

The electrical conductivity of raw beef loin experiment involved numerous parameters (n=6 loins×6 steaks/loin×1 replication/steak=36 total observations). All samples were individually placed in a separate Ziploc bag and emulsified. Emulsification of each 50 g raw beef loin sample was conjoined with 50 ml of deionized water. The emulsifications were then placed into a silicon vessel illustrated in FIG. 2. The meat slurry was placed into the silicon vessel, and two electrodes were placed on the opposite ends for an EC reading. An EC measurement appeared after 60 seconds through the applied rate of 2 readings per second. Voltage and current flow were monitored through a multimeter (16040T True RMS Multimeter, Southwire Tools & Equipment). Between the sample readings, all materials and equipment exposed were rinsed with deionized water after every trial run. At the end of the experiment, all equipment was cleaned thoroughly before initiating any other EC experiments.

The same loins used for color measurements were used for electrical conductivity analysis. The fresh electrical conductivity analysis was needed for testing ionic strength in raw samples to identify the ionic strength of possible water forms found in fresh beef loins.

### Example 2.4. Experimental Design for Tenderness in Cooked Loins

Beef loin samples used in this experiment were cooked for the evaluation of tenderness. Steaks placed in the walk-in cooler were taken out and left to rest approximately one hour. Steaks went through EC raw evaluation, and then the beef loins were weighed and placed onto a meat metal tray by date, by loin and steak number. Once allocated, beef loins were placed into a multi-purpose smoker (UltraSource Grand Prize™ 3) with an internal temperature set at 165° F. (74° C.). Beef loins were cooked for approximately three hours. Subsequently, steaks were allowed to rest for 30 minutes before storage. Afterward, steaks were weighed using a food-grade scale and placed into gallon-sized Ziploc bags with their corresponding loin and steak number, arranged into a plastic white bin and stored in a walk-in cooler overnight at 34° F. (1° C.) overnight.

The beef loins were taken out and placed onto a square cut butcher paper labeled with its loin and steak number. Steaks were allowed to rest approximately one hour before coring. After rest, steaks were cored six times using a handheld coring device and cores weighed about 50 grams each. The Warner-Bratzler Shear Force (WBSF) device calculated shear force values specified through (G-R Manufacturing, Tall Grass Solutions, Manhattan, Kans.) to provide tenderness values. Six cores/steak, weighing at 50 g, provided consistency for electrical conductivity procedures. The tenderness portion of this experiment consisted of (n=6 loins×6 steaks×6 cores/steak=216 total observations). When shear force values were completed, the core samples were placed on brown butcher paper labeled with loin and steak number. After all the core samples were completed and EC determinations were collected, the same loins were used for color measurements and cooked for tenderness analysis. The tenderness analysis was needed for testing the strength in cooked loins to provide an improved assessment for beef quality grading.

### Example 2.5. Experimental Design for Electrical Conductivity of Cooked Steaks

Beef loins procured from a commercial producer were used for tenderness measurements and subsequently for EC determinations. The ionic strength was evaluated from each of the six core samples, with a total weight of 50 g from each steak (N=6 samples×6 steaks=36 per source) used for EC evaluations. The core samples were labeled by loin steak number using white/brown butcher paper and a permanent marker. Ambient temperature was also recorded to ensure temperature calibration. Cooked 50 g core samples and 50 ml of deionized water were emulsified using Ninja BL 456 blinder, which created a dilution factor of 2.

Next, the colloidal solution was dispersed into the silicon vessel. Once the sample was placed into the vessel, two holes placed on opposite ends served to hold two copper electrodes of the digital multimeter and prevent leakage (16040T True RMS Multimeter, Southwire Tools & Equipment). The preparations in the silicon vessel and the alignment with the two electrodes were completed simultaneously. An EC reading, using units (a), was recorded for 60 seconds with the application of the rate of 2 readings per second. After each source was read, all materials and equipment exposed to the preparations were cleaned and bleached to prevent any contamination for the next sample.

The same loins used for color measurements were cooked in order to complete cooked electrical conductivity analysis. The cooked electrical conductivity analysis was used for appraisal of the ionic strength in cooked samples to evaluate the ionic strength of possible water forms in cooked beef loins.

### Example 2.6. Statistical Analysis

All data collected in this study were analyzed through SAS software and Microsoft Excel 2016 procedures. Applicable procedures for statistics included Pearson correlation coefficient to examine color variables, tenderness and electrical conductivity for water configuration predictions. The use of one way and two way ANOVA were completed to identify any differences of means.

The statistical design for experiments were as follows: color variables consisted of a complete block design (blocks were steaks within a strip loin); electrical conductivity consisted of a block design (blocks were steaks within a strip loin) in both fresh and cooked loins; and electrical conductivity in beef jerky consisted of repeated measures from one source. Tenderness in cooked loins encompassed a completely randomized design.

### Example 2.7. Beef Jerky Electrical Conductivity Determinations

The results for EC determinations of commercial beef jerky were analyzed using one way ANOVA to evaluate EC values statistically. The results indicated (Mean=106.87, SD=42.85, SEM=5.05 and CV=40.10). Ingredients in the commercial beef jerky source comprised of food additives that contained ions, reflecting high EC values and indicating high ionic concentration.

### Example 2.8. Color Attribute/Component Evaluations

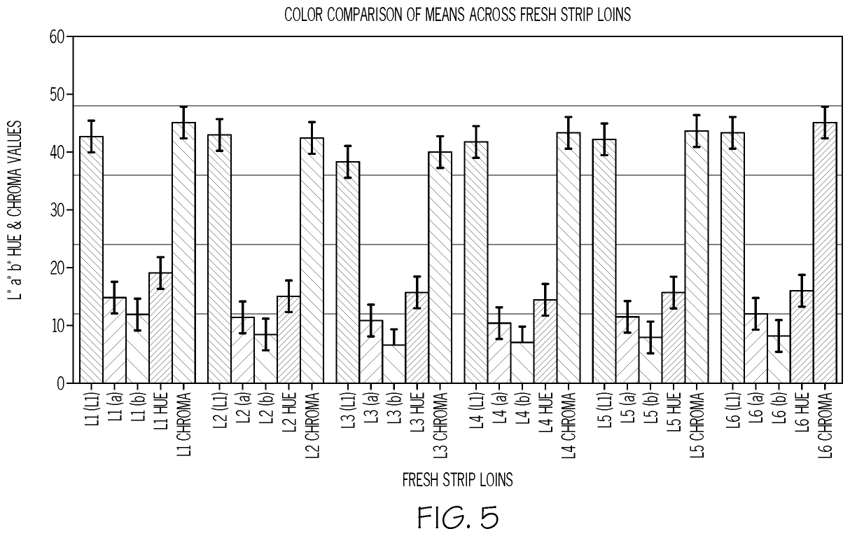

All color data were analyzed using one way ANOVA and Pearson correlation coefficients to dictate further how color variables correlated within each other. An evaluation of color is demonstrated in FIG. 5, which shows the color comparison of means across fresh strip loins with color variables of chroma, hue, L*, a*, and b*. The figure held similar color values across means for chroma, hue, L*, a*, and b*. Still, when compared to different color values, there was a variation among all loins, especially chroma, hue, and L* value. Overall, the correlations indicate that there was a high degree of variation in color variables across loins in this study.

### Example 2.9. Tenderness Evaluation for Cooked Strip Loins

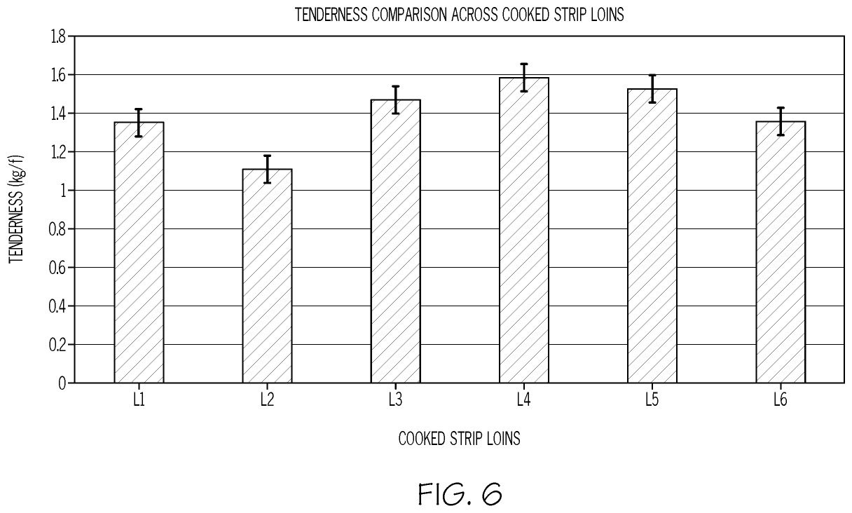

The results for tenderness are indicated by Warner Bratzler Shear Force (WBSF) values, which are individually identified as strip loins 1-6, which were cooked. A complete visual representation of tenderness between cooked strip loins 1-6 is presented in FIG. 6. Strip loin 4 showed a high mean WBSF value of (Mean=1.59 kg/f) and strip loin 2 had a low mean WBSF value of (Mean=1.1 kg/f). Other factors considered for analysis were overall (Mean=1.39, F=9.74, df=5). All analyses were set to alpha (P<0.05), which showed a significant difference between strip loins at (P<0.001), indicating there was a high degree of variation in tenderness across loins in this study.

### Example 2.10. Relationship of Fresh Electrical Conductivity to Color

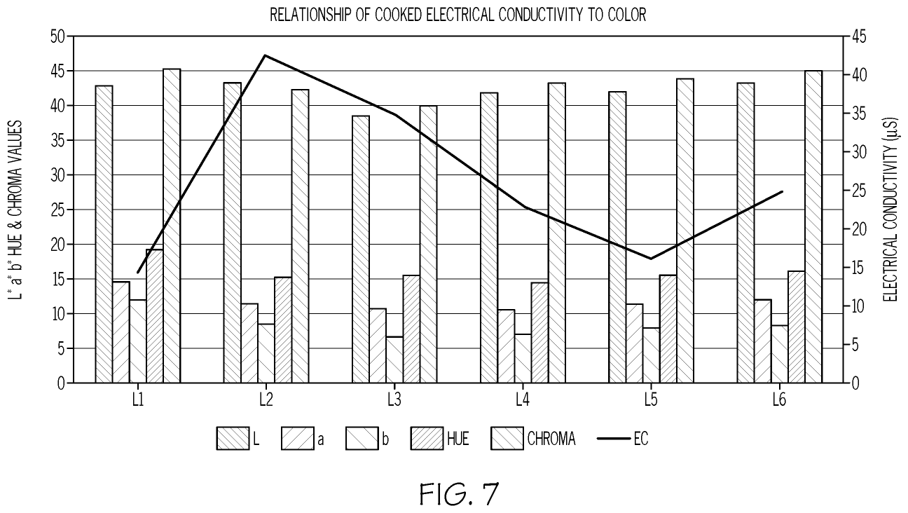

The data displayed in FIG. 7 is from fresh strip loins correlating EC to color. The fresh EC is indicated by a line and the color variables are indicated by columns. This illustration indicates loin 2 peaked at a higher EC value (42.52) than other loins. At the peak, the EC declined to a low for loin 5 (16.05) then increased at loin 6 (24.70). Loins 1 & 5 contained the lowest EC values at (14.37) and (16.05). All analysis was completed at (P<0.05), and showed a negative relationship among these variables.

The data in FIG. 7 shows the relationship with color and fresh EC values. This relationship suggests that values L*, a*, b*, hue and chroma values have a negative correlation with fresh loin EC. The L* value was not different (P=0.98), a* value was different (P=0.05), b* not different (P=0.10), hue not different (P=0.46) and chroma not different (P=0.84). The analysis was completed at (P<0.05). As the color variables increase, the electrical conductivity decreases in fresh beef loins. Fresh electrical conductivity may indicate the color depiction in beef loins.

### Example 2.11. Relationship of Fresh Electrical Conductivity to Tenderness

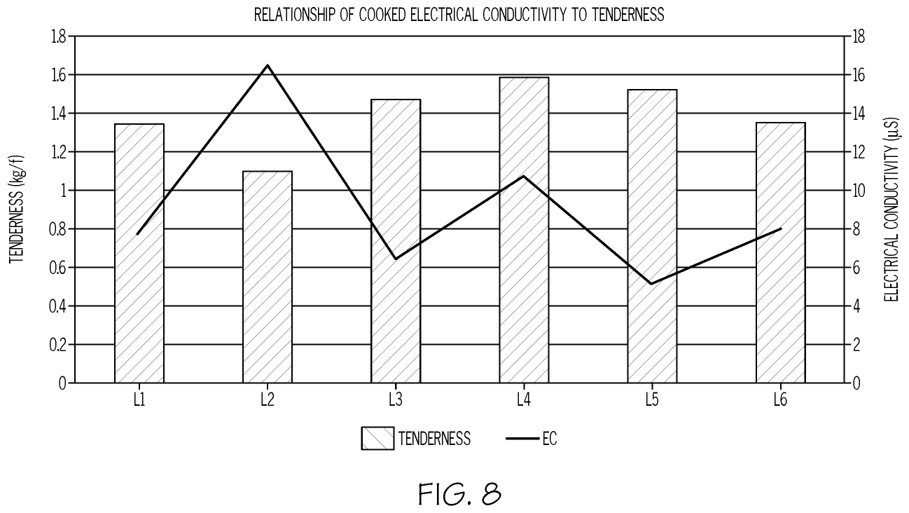

The data displayed in FIG. 8 is from fresh strip loins correlated with electrical conductivity to color. The fresh EC is indicated by a line and tenderness is indicated by columns. This illustration indicates loin 2 peaked at a higher EC value (42.52) than other loins. A decline in EC began from loin 2 (42.52) to loin 5 (16.04) then, began an increase at loin 6 (24.70). Loins 1 and 5 contained the lowest EC values at (14.37) and (16.05).

### Example 2.12. Relationship of Cooked Electrical Conductivity to Tenderness

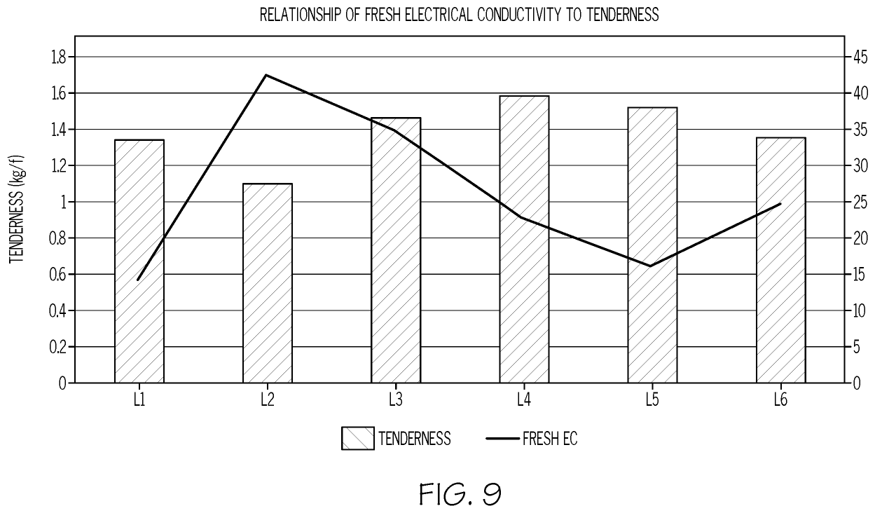

The data displayed in FIG. 9 shows the correlation of EC to tenderness. The cooked EC is indicated by a line and tenderness is indicated by columns. This illustration shows loin 2 peaked at a higher EC value (16.47) than the other loins. At the peak, the EC drops in loin 3 (6.42), then rises in loin 4 to (10.73), decreases again in loin 5 (5.17) lastly, rises in loin 6 (7.99). Loins 1 and 6 show similar EC values (7.99 and 7.78). All analyses were completed at (P<0.05), and showed a negative relationship among these variables.

### Example 2.13. Relationship of Cooked Electrical Conductivity to Color

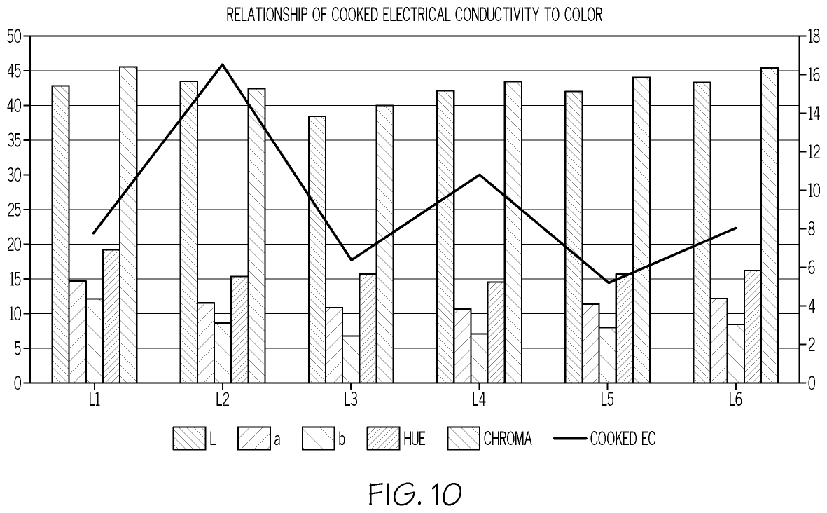

The data displayed in FIG. 10 shows the correlation of EC to color in cooked loins. The cooked EC is indicated by a line and color variables are indicated by columns. This illustration indicates loin 2 peaked at a higher EC value (16.47) than the other loins. At the peak, the EC drops in loin 3 (6.42), then rises in loin 4 to (10.73), decreases again in loin 5 (5.17) lastly, rises in loin 6 (7.99). Loins 1 and 6 show similar EC values (7.99 and 7.78). The data indicate that there may be a way to measure redness, yellowness and the color description for beef loins using cooked electrical conductivity.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of evaluating a meat sample, said method comprising:
measuring electricity conducted from water in the meat sample; and
correlating the measurement to a property of the meat sample.

2. The method of claim 1, wherein the water is derived from at least one water form selected from the group consisting of free water, immobilized water, bound water, or combinations thereof.

3. The method of claim 1, wherein the measuring step comprises measuring micro Siemens of electricity conducted from water in the meat sample.

4. The method of claim 1, wherein the measuring step comprises measuring electron flow from resulting water in the meat sample.

5. The method of claim 1, wherein the measuring step comprises measuring an electrical conductivity of the meat sample.

6. The method of claim 5, wherein the meat sample is placed in a container comprising water prior to the measuring step.

7. The method of claim 6, wherein the water is deionized water.

8. The method of claim 5, wherein the container is connected to one or more electrodes for measuring electrical conductivity.

9. The method of claim 1, wherein the meat sample is selected from the group consisting of beef, poultry, fish, pork, and combinations thereof.

10. The method of claim 1, wherein the meat sample is beef.

11. The method of claim 1, wherein the correlating comprises comparing the measurement to known properties associated with the measurement in similar meat samples.

12. The method of claim 11, wherein the comparing comprises comparing the measurement to a database that includes the known properties.

13. The method of claim 1, wherein the correlating comprises:
correlating the measurement to a fat content of the meat sample;
correlating the fat content to a quality grade of the meat sample; and
utilizing the correlated quality grade to predict a meat sample property.

14. The method of claim 1, wherein the correlating comprises the utilization of an algorithm to correlate the measurement to one or more properties of the meat sample.

15. The method of claim 1, wherein the property of the meat sample is a color of the meat sample.

16. The method of claim 15, wherein the color of the meat sample represents a color score of an uncooked meat sample.

17. The method of claim 1, wherein the property of the meat sample is tenderness of the meat sample.

18. The method of claim 17, wherein the tenderness of the meat sample represents the tenderness of the meat sample in cooked form.
